(19) United States
(12) Patent Application Publication
(10) Pub. No.: US 2017/0290526 A1
(43) Pub. Date: Oct. 12, 2017

(54) TELECENTIVE SPIROMETER

(71) Applicant: Hormozd Bozorgchami, Portland, OR (US)

(72) Inventor: Hormozd Bozorgchami, Portland, OR (US)

(73) Assignee: Oregon Health & Science University, Portland, OR (US)

(21) Appl. No.: 15/481,258

(22) Filed: Apr. 6, 2017

Related U.S. Application Data
(60) Provisional application No. 62/319,715, filed on Apr. 7, 2016.

(51) Int. Cl. A61B 5/087; A61B 5/00; G08B 21/18

(52) U.S. Cl. CPC A61B 5/087; G08B 21/18; A61B 5/4833; A61B 5/0015

(57) ABSTRACT

Systems and methods to encourage a patient to use an incentive spirometer (IS) device according to the schedule prescribed by a clinician are described. The systems and methods for a clinician-programmable IS device that can automatically monitor patient usage, and, in the event of non-compliance, effect an automatic shutdown of an entertainment device, such as a television in the patient's room. The programmable IS device can provide feedback to the patient when the device is used properly, warnings when the device is under-utilized, and can also save information regarding IS usage history to a storage medium for later review and analysis.

### CROSS-REFERENCE TO RELATED APPLICATION

This application claims priority to U.S. Provisional Patent Application No. 62/319,715 filed Apr. 7, 2016, which is incorporated herein by reference in its entirety as if fully set forth herein.

### FIELD OF THE DISCLOSURE

The present disclosure relates to the field of incentive spirometers, and, more specifically to systems and methods to increase patient compliance in using them as directed.

### BACKGROUND OF THE DISCLOSURE

Postoperative pulmonary complications (PPCs) are common problem in hospital environments, particularly for patients recovering from abdominal, cardiac, and thoracic surgery. These complications may include pneumonia, altelectasis (partial or complete collapse of the lung due to deflation of alveolar sacs), acute respiratory failure, and tracheobronchitis. PPCs increase morbidity and mortality rates in patients, extend the length of hospital stay, and drive associated healthcare costs upward (Branson R et al, Resp Care 58, 1974-84 (2103); Carvalho C et al, Rev Bras Fisioter 15, 343-50 (2011); Popssa S et al, Rev Port Pneumol 20, 69-77 (2014); incorporated by reference herein). It is generally accepted that PPCs are exacerbated by the disruption of normal patterns of breathing which, in turn, diminish lung capacity. Examples of disrupted breathing patterns include prolonged shallow breathing, monotonous tidal volume, and an absence of the spontaneous deep breaths which normally occur every 5 to 10 minutes in healthy individuals (e.g., sighing). These compromised ventilation patterns have multiple contributory factors including anesthesia effects, opioid analgesia, muscle dysfunction, and post-operative pain. To overcome negative effects of compromised lung ventilation, deep breathing-based interventions that promote lung re-expansion have been recommended as strategies to prevent PPCs and restore ventilatory function.

The incentive spirometer (IS) is a medical device designed to promote the rehabilitation of lung ventilation following surgery. Developed in 1970, the purpose of the IS is to encourage the patient to take periodic deep breaths to stimulate the expansion of collapsed airways and alveoli (Bartlett R et al, Surg Forum 21, 222-4 (1970); incorporated by reference herein). The IS provides a visual indication of the inspirational effort exerted when the patient performs a maximal sustained inspiration (inhalation) through the device. The visual indication may indicate, for example, that a desired flow rate or a target volume of air has been achieved on a given inspirational effort. Such visual feedback is intended to provide an “incentive” or goal for the patient to try to meet when using the device.

In particular embodiments, the IS may take the form of a low-cost disposable plastic cylindrical column housing a float. This float can be made to rise when air is inhaled through a tube attached to the column. The cylindrical column is often marked with graduations representing target flow rates or volumes that the patient aims to achieve. During an inhalation effort the patient first raises the float to the target level, and then attempts to maintain it at that level for as long as possible by sustaining the inspiration. Operating the IS in this manner promotes sustained maximal inspiration (inspiration to total lung capacity for the longest possible time), expanding the lungs and opening alveolar sacs. This intervention decreases the likelihood that the patient will develop atelectasis or other pulmonary complications.

Once the patient has been instructed in the use of the IS, and the targets and frequency of use specified (usually several inhalations per hour), he or she is expected to operate the IS on a voluntary basis with minimal oversight. However, patient compliance with scheduled use of the IS can be poor, with some reports suggesting that as few as 10% of patients use the IS as directed. This non-compliance, in turn, is believed to contribute to PPCs which negatively impact patient outcomes, lengthen hospital stays, and increase costs. Thus, there is a need for a more compelling incentive mechanism to encourage patient usage of the IS.

### SUMMARY OF THE DISCLOSURE

The present disclosure is directed to systems and methods to increase compliance in the usage of IS devices. The inventor herein has observed, in the course of visiting patients' rooms, that the prescribed IS device is often placed out of reach of the patient and not being utilized. The inventor has further noted that patients typically have access to, and regularly use, entertainment modalities (e.g., televisions, radios, gaming systems) that have been placed in their rooms to provide entertainment during the post-surgical recovery period. These entertainment modalities are often remote controlled via an interface that is attached to the patient's bed. Thus, one aspect of the disclosure is the integration of a standard IS device with the entertainment system such that the power to the entertainment modality can be interrupted if the IS is not used periodically (e.g., television entertainment is withheld when the patient does not comply with the prescribed IS usage schedule). In this manner, the incentive mechanism for the patient to use the IS device is extended beyond the simple visual feedback typically employed in such devices (e.g., a floating buoy housed in a cylindrical column that the patient controls by breathing through the spirometer) to further include withholding of an activity that the patient finds desirable (e.g., watching television). The inventor has further observed that patients are often accompanied by family members in their rooms during recovery, and that these family members also engage with the entertainment modalities with the recovering patient. An aspect of the disclosure is that non-usage of the spirometer resulting in interruption of the entertainment modality will also serve as an incentive for family members to encourage the patient to use the IS device, so that entertainment is not interrupted.

The system disclosed herein includes components to monitor and detect usage (or non-usage) of the IS device, and components to interrupt one or more entertainment modalities (e.g., viewing of the television). An aspect of the disclosure is that can be implemented as an attachment or modification to IS devices currently available. Specifically, the components to monitor usage can be inserted in-line with the breathing tube component of a standard IS, such that a redesign of existing IS devices is not necessary. In particular embodiments, this component can utilize a differential pressure sensor to detect airflow as an indicator of patient usage. Alternatively, an anemometer or turbine-based device can be employed in this component to detect air velocity as an indicator of device usage.

A further aspect of the disclosure, as described above, is a programmable processing unit which monitors the frequency of usage of the IS device and, if necessary, effects a shutdown of an entertainment modality to which it is interfaced. In particular embodiments, a programming interface can be provided to allow a clinician to specify usage frequency parameters to which the patient must adhere, for example, a target number of inhalations to be performed through the IS device over a given timespan. In particular embodiments, the processing unit can provide a reminder signal to the patient to use the IS in advance of a shutdown, for example, through an indicator light, multicolor LED, emitted sound, or other appropriate cue. This monitoring unit can further contain functionality to record patient usage data, such as continuous time history of IS usage, time-stamped inspiration efforts, duration of inhalation for a given effort, number of consecutive inspirations within a prescribed time interval, or similar usage statistics, which can be stored for later review.

The entertainment modality shutdown component of the electronic unit can be implemented in a number of ways, all aimed at disrupting access to entertainment. In particular embodiments, the electronic unit can interface with the existing remote control for the entertainment modality (e.g., television) such that the entertainment modality remote acts as a entertainment modality shutoff unit by emitting a “turn off” signal when required. Alternatively, the electronic unit can be configured to send its own “turn off” signal directly to the entertainment modality, much like the universal remote control systems that are known in the art. In particular embodiments an entertainment modality shutdown system incorporates an electrical component that disrupts the power supplied to the entertainment modality using, for example, a relay switch. Such an entertainment modality shutoff unit can reside, in particular embodiments, between the entertainment modality and a power source (e.g., wall power outlet) where it can receive a shutoff signal wirelessly and effect a hard shutoff of the entertainment modality's power. Such an implementation has the advantage that power to the entertainment modality is physically interrupted and cannot simply be restored using a remote control.

In all of the aforementioned embodiments, signals to turn off the entertainment modality may be communicated using direct wired connections, wireless communication protocols, and hybrid wired-wireless implementations. Examples of wireless protocols include radio frequency signals, infrared signals, Bluetooth, and wifi-based protocols, including those used in home-automation systems.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE SEVERAL VIEWS OF THE DRAWINGS

FIG. 1 is a drawing of a typical incentive spirometer.

FIG. 2 is an exemplary system diagram for a telecentive spirometer.

FIG. 3 is a system diagram for a prototype telecentive spirometer.

FIG. 4A is a picture of a venturi tube in cross section.

FIG. 4B is a rendering of a 3D CAD model of the venturi tibe of FIG. 4A.

FIG. 4C is a picture of a venturi tube placed in-line with a spirometer inhalation tube.

FIG. 5 is a state diagram describing the software architecture used to program the microcontroller in the prototype telecentive spirometer disclosed herein.

FIG. 6 is a flowchart of the operations performed when the telecentive spirometer microcontroller is in the Initialize state.

FIG. 7 is a flowchart of the operations performed when the telecentive spirometer microcontroller is in the Idle state.

FIG. 8 is a flowchart of the operations performed when the telecentive spirometer microcontroller is in the Measure Inhalation state.

FIG. 9 is a flowchart of the operations performed when the telecentive spirometer microcontroller is in the Evaluate Inhalation state.

FIG. 10 is a flowchart of the operations performed when the telecentive spirometer microcontroller is in the Evaluate Use state.

FIG. 11 is a flowchart of the operations performed when the telecentive spirometer microcontroller is in the Error state.

FIG. 12 is a block diagram of the prototype shutoff unit to be used with the telecentive spirometer.

FIG. 13 is a flowchart of the operations performed by the shutoff unit microcontroller.

### DETAILED DESCRIPTION

The following description is directed to systems and methods for increasing patient compliance in the use of prescribed IS devices. Patients are motivated to comply by the negative consequence of loss of entertainment (e.g., television viewing) when the IS device is not used according to physician orders. In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents. The description may use the terms “embodiment” or “embodiments,” which may each refer to one or more of the same or different embodiments.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in improved patient compliance as described herein.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

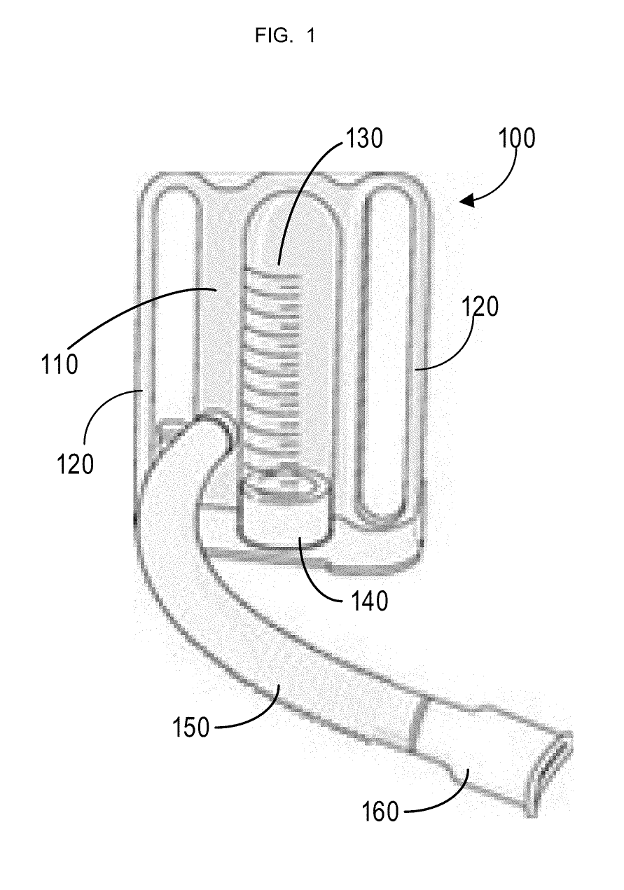

As described previously, IS devices are regularly prescribed as part of a postsurgical therapy regimen to promote the recovery of lung function and prevent postoperative complications such as atelectasis or pneumonia. Disclosed herein are systems and methods to augment a standard IS device so that patient usage of the IS is monitored and a corrective action initiated in the event that the IS goes unused or under-used for a given period of time. As disclosed herein, the corrective action entails the removal of patient access to a form of entertainment (e.g., television viewing, radio listening, drawn curtains for outside viewing) as a means to motivate the patient to use said IS device. FIG. 1 shows an example of a standard disposable IS device 100 that might be supplied to a patient recovering from surgery. As depicted, the IS device 100 includes a main body 110 with handles 120 on the left and right sides, and an integrated graduated hollow cylindrical column 130 within which a float 140 is housed. An inhalation tube 150 is connected to the main body 110 of the IS device. The float 140 can be made to rise within the graduated column 130 when the patient draws a breath through the inhalation tube 150 via a mouthpiece 160 connected to the tube 150.

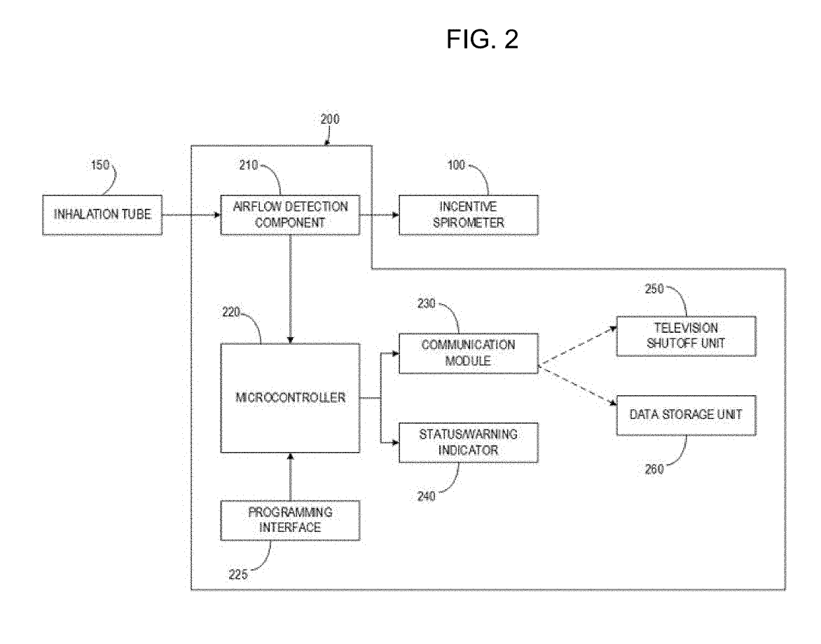

The disclosed system, herein termed a “telecentive spirometer,” is incorporated into a standard IS device such that the function of the IS is augmented to include capabilities for monitoring device usage and for interrupting entertainment in the event that the device is not being used as prescribed. FIG. 2 shows a schematic embodiment of a telecentive spirometer system 200, wherein an airflow detection component 210 is connected in-line with the inhalation tube of an IS device (for instance, between the inhalation tube 150 and the main body 110 of a standard IS device 100, or between the mouthpiece 160 and the inhalation tube 150). The airflow detection component 210 includes components capable of transducing inhalation-induced air flow into an electrical signal that is detectable by a microcontroller 220 or other circuitry acting as a processing unit that has been programmed to monitor output signals from the airflow detection component 210. The microcontroller 220 also contains functionality to track, for example, the quality of an inhalation effort or the number of ‘good’ inhalations over a given time interval, and output a signal to a status/warning indicator 240 to alert the patient that a given amount of time has elapsed. The microcontroller can also be accessed through a programming interface 225 to allow a clinician to specify program parameters (e.g., number of inhalations to be performed by the patient over a given length of time). This programming interface 225 can also allow access to parameters governing the detection and analysis of signals received from the airflow detection component 220, so that device sensitivity can be adjusted to ensure proper detection of device usage events (for example, one might adjust threshold values associated with signal amplitude and signal duration to specify requirements for an inhalation event to qualify as ‘good’ and, therefore, be counted as a use of the device). In particular embodiments, this programming interface can include physical input components on the device 200 such as switches, dials, buttons, touchscreen interfaces, and keypads. In particular embodiments, the programming interface can include access to the microcontroller 220 through a wired or wireless connection to a device having a user interface, for example, a computer, a tablet, a remote control, or a mobile computing device such as a mobile telephone.

The microcontroller 220 further contains functionality to pass signals to one or more communication modules 230 configured to particular embodiments, this communication module 230 is designed to convey a shut-off instruction to an entertainment modality shutoff unit 250 configured to accept such instruction. In particular embodiments the communication module 230 can be designed to convey patient usage data to a data storage unit 260 for archival purposes or for further processing and analysis. This data storage unit 260 can be physically integrated into the telecentive spirometer device or exist separately and configured to receive data through the communication module 220. Examples of patient usage data include the time history of signals from the airflow detection component 210, time-stamped event data, number and durations of inhalations within a given time period, summary statistics of device usage, or other relevant data. In particular embodiments, the type of patient usage data to be output from the system 200 and the destination for that data can be specified though a programming interface 225 so that the type of output data may be tailored to clinician or patient needs.

In particular embodiments the airflow detection component 210 includes any suitable device for measuring air flow within a tube and transducing that measurement into an electrical signal. Examples of such devices include anemometer-based devices which measure temperature change or heat transfer from an electrically heated wire, or a turbine-based device configured to detect the number of revolutions per unit time under flow conditions (e.g., using an optoelectronic counter to detect light interruptions at each turn of the turbine blades). Alternatively, the airflow detection component 210 includes a device that induces a pressure change across a given axial length during air flow in combination with another component capable of detecting and transducing said pressure change into an electrical signal. In particular embodiments described below, such a pressure-based airflow detection system may be implemented by connecting the ports of a differential pressure sensor to the pressure taps of a venturi tube placed in-line with the inhalation airflow path. Further examples of pressure-based flow detection systems include Fleish and Lilly pneumotachometers, wherein an element with known fixed resistance is placed in the flow path to produce a pressure drop across said element that is proportional to flow velocity.

In particular embodiments, the entertainment shutoff procedure is enacted by the microcontroller 220 component first analyzing the time history of the spirometer usage (e.g., by a timer function), and then, if necessary, sending an appropriate shutoff signal to the entertainment shutoff unit 250 via a communication module 230. In particular embodiments, the communication of this signal can be implemented using a hardwire connection, for example, between the telecentive spirometer's communication module 230 and an entertainment modality remote control or to a separate entertainment modality shutoff unit. In addition, wireless methods can be used by the communication module 230 to convey shutoff signals from the telecentive spirometer system. Examples of such wireless signal protocols include radio frequency signals, infrared signals, Bluetooth, and wifi-based protocols, including those used in home-automation systems. In particular embodiments, shutoff signals can be sent directly to the entertainment modality, to a remote control associated with the entertainment modality, or to an intermediate device capable of disrupting operation of the entertainment modality (e.g., by disrupting the power to the entertainment modality or disrupting the entertainment modality signal). In particular embodiments described below, the Bluetooth wireless communication protocol is used to transmit a shutoff signal to a relay switch-based device which resides between the entertainment modality and the wall power outlet, enabling a method to physically interrupt and restore power to the television based on spirometer usage.

Prior to sending a shutoff signal, the microcontroller 220 can also be configured, in particular embodiments, to provide a reminder to the patient to use the IS through a status/warning indicator 240 subsystem. This patient reminder system can be implemented as hardware integrated into the telecentive spirometer, for example, as an indicator light, a multicolor LED, a countdown timer, or a speaker capable of emitting a warning sound. Alternatively, the status/warning indicator 240 can be configured to communicate with an external device to provide a reminder to the patient to use the IS. Examples of such communication can include a message displayed on the television, a text message sent to a cellphone, or other appropriate notification.

It is to be understood that the configurations and/or approaches described herein are exemplary in nature, and that these specific embodiments or examples are not to be considered in a limiting sense, because numerous variations are possible. The subject matter of the present disclosure includes all novel and nonobvious combinations and subcombinations of the various processes, systems and configurations, and other features, functions, acts, and/or properties disclosed herein, as well as any and all equivalents thereof.

### Examples

The following examples are illustrative of the disclosed systems and methods. In light of this disclosure, those skilled in the art will recognize that variations of these examples and other examples of the disclosed systems and method would be possible without undue experimentation.

An example embodiment of a telecentive spirometer is disclosed below. Briefly, the design utilizes a venturi tube connected in-line with the inhalation tube of the IS to induce a pressure differential during a sustained inhalation effort. An electronic differential pressure sensor attached across the ports of the venturi tube transduces airflow into an electrical signal which is sent to a microcontroller. Two Bluetooth modules are incorporated into the design: one to communicate with a cell phone device, and a second to communicate with a television shutoff unit. The shutoff unit is a standalone component that resides between an electrical wall outlet and the television. The shutoff unit houses a circuit including a relay switch, a microcontroller, and a Bluetooth module configured to either disrupt or restore electrical connection to the wall outlet. The system is programmed to monitor device usage (i.e., inhalation effort by the patient) and if the spirometer device is not used within a given period of time (e.g., at nine minutes), emit a warning beep. After an additional period of time (e.g., at ten minutes), the system shuts off the television.

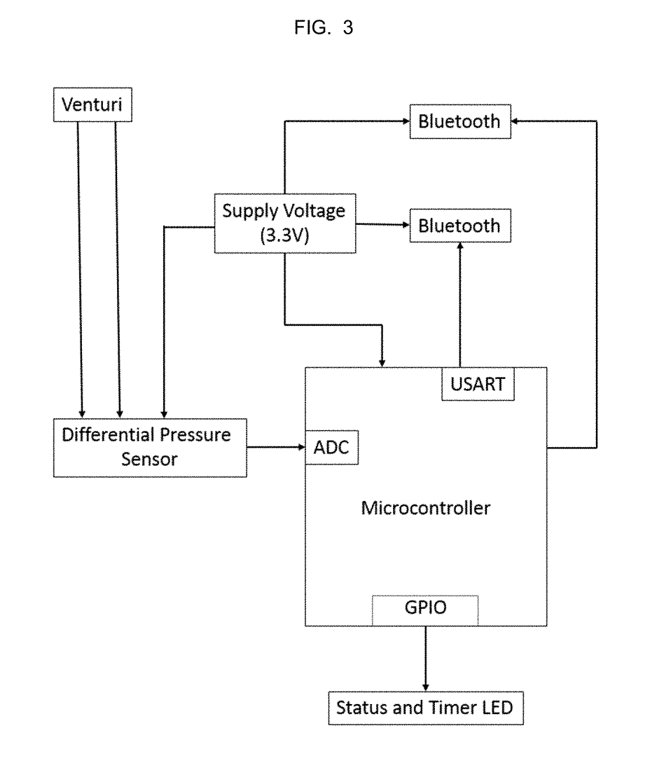

Telecentive Spirometer System Architecture. FIG. 3 shows a system diagram for a prototype telecentive spirometer described herein. A differential pressure sensor (MPXZ7007DP-ND) receives two pressure inputs from a custom-fabricated venturi tube (see below) and transduces the differential pressure into a voltage signal and passes that signal to a microcontroller. In the prototype described herein, the ATXMEGA64A4U microcontroller was chosen specifically for its advanced ADC (analog-to-digital converter) features, multiple timer capability, GPIO (General-purpose input/output) functionality, and the multiple USARTs (Universal Synchronous Asynchronous Receiver Transmitters) that are available to communicate with multiple Bluetooth Modules. The microcontroller is responsible for taking samples of the input from the differential pressure sensor and converting them to digital signals via the ADC. The microcontroller is also responsible for sending RS-232 signals to the Bluetooth modules. Lastly, the microcontroller provides output to the Status and Timer LED.

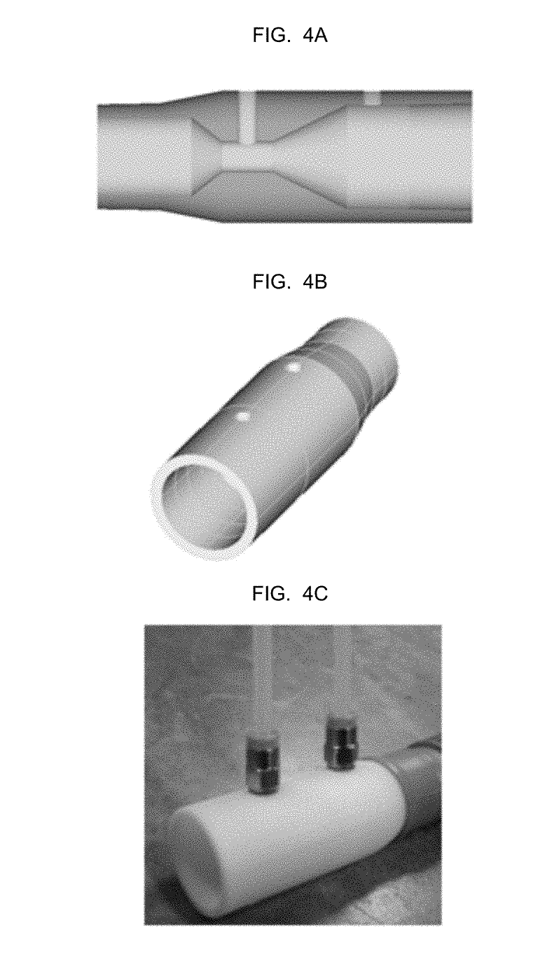

Venturi Tube for Airflow Detection. FIGS. 4A-4C show an example of a venturi tube that was custom fabricated for inline placement with the inhalation tube of the spirometer. As shown in the computer-aided-design models of FIG. 4A and FIG. 4B, the venturi tube was designed to accommodate two pressure ports for connection to the MPXZ7007DP-ND differential pressure sensor. FIG. 4C shows a picture of the fabricated venturi tube inserted into one end of the spirometer inhalation tube.

Status and Timer LED. The microcontroller drives one multicolored LED to visually inform the patient of the status of the unit. The LED also provides visual warnings of when to use the spirometer, as well as an indication if the use was a GOOD use or a BAD use. In the prototype describe herein, six separate LED states were programmed to provide feedback to the patient. A brief description of these states is provided in Table 1.

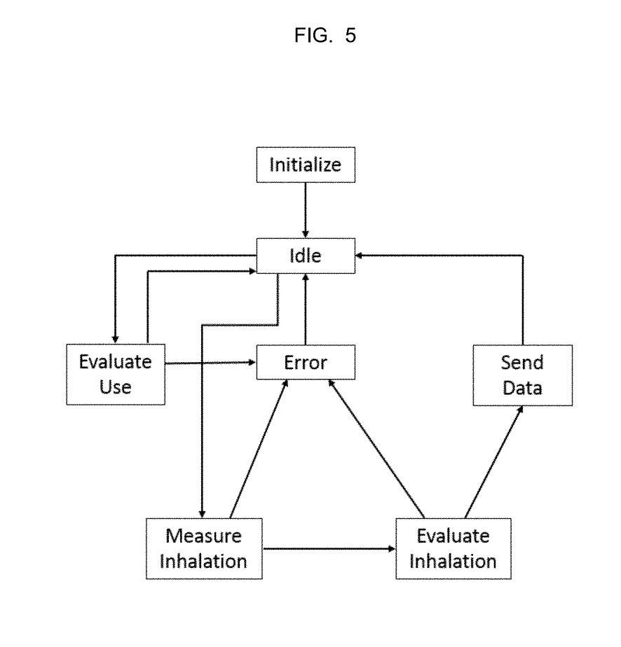

Software Architecture. A state machine approach was adopted to program the microcontroller functions for the prototype described herein. FIG. 5 shows the state diagram used in the disclosed prototype. Briefly, the main software loop instantiates the data structures and creates pointers to these structures. A watchdog timer is also evaluated. The watchdog signal is sent to the shutoff unit every two seconds. This ensures that if power is removed from the main spirometer unit, the watchdog will fail, and the shutoff unit will remove power from the TELEVISION. After evaluating the watchdog timer, the state machine will be updated and actions will be performed based on the state. The seven states depicted in FIG. 5 are described below.

TABLE 1
Tri-Color LED Status Descriptions

| LED Color | State | Meaning |
|---|---|---|
| Green | Solid | Power ON |
| | Blinking | GOOD Inhale |
| Yellow | Solid | Noise/Tube is Backwards |
| | Blinking | 5 minute Warning |
| Red | Solid | Shutoff Signal Sent |
| | Blinking | 1 minute warning/BAD Inhale |

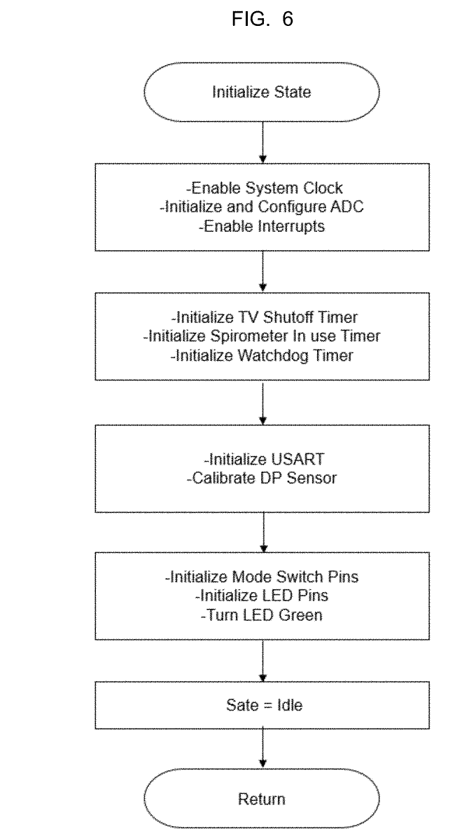

Initialization State. The Initialization state configures peripherals and prepares the spirometer for use by performing a number of actions as shown in the flowchart of FIG. 6. These action include enabling the system clock and interrupts, and initializing timers associated with television shutoff, spirometer “in use,” and watchdog timer. The ADC, USART, and GPIO functions of the microcontroller are configured and enabled to accept input from the differential pressure sensor, communicate with the Bluetooth modules, and receive inputs from the Spirometer Mode Switch, respectively. Finally, a function is called to calibrate the differential pressure sensor and the LED status is initialized to green. After Initialization is complete, the state is changed to the Idle state.

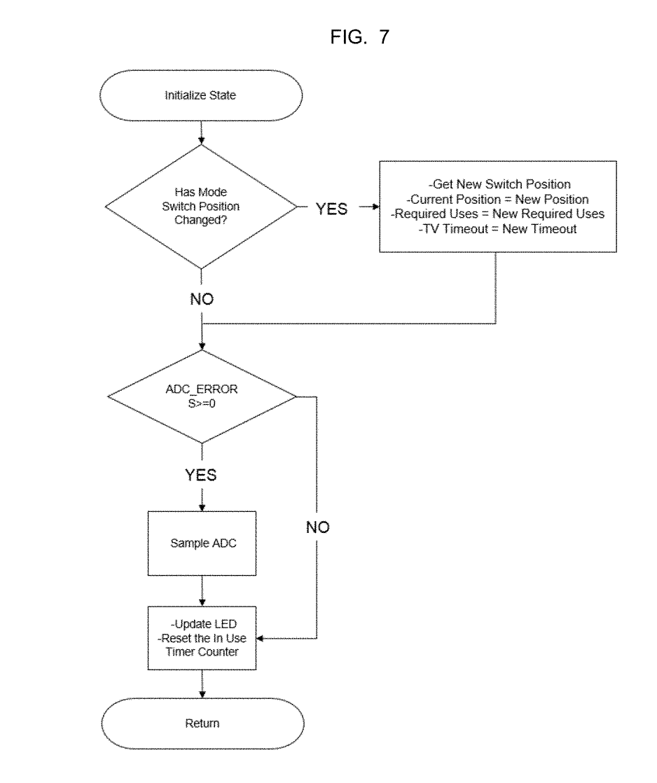

Idle State. FIG. 7 shows a flowchart of the Idle state of operation. The Idle state evaluates whether the Spirometer Mode switch has changed. If so, the Use parameters are changed accordingly. The Idle state also monitors the ADC to determine if the patient is using the spirometer. The status LED is updated in the idle state to inform the patient of 5 minutes (LED blinking yellow) and 1 minute (LED blinking red) remaining to complete the required spirometer usage before power is removed from the television.

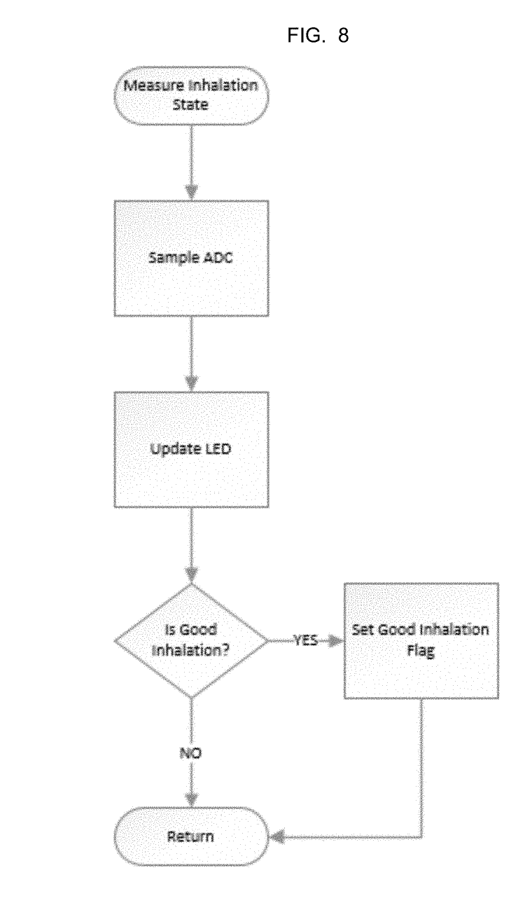

Measure Inhalation State. FIG. 8 shows a flowchart of the Measure Inhalation state. This state is active if the input from the DP sensor has exceeded the threshold set to determine if the spirometer is in use. The ADC is continuously monitored in this state while the patient is inhaling. If the patient meets the minimum requirement of a successful inhalation (e.g., inhalation for greater that 0.5 seconds) then a flag is set marking the use as a good inhalation. The LED is turned off during the measure inhalation state.

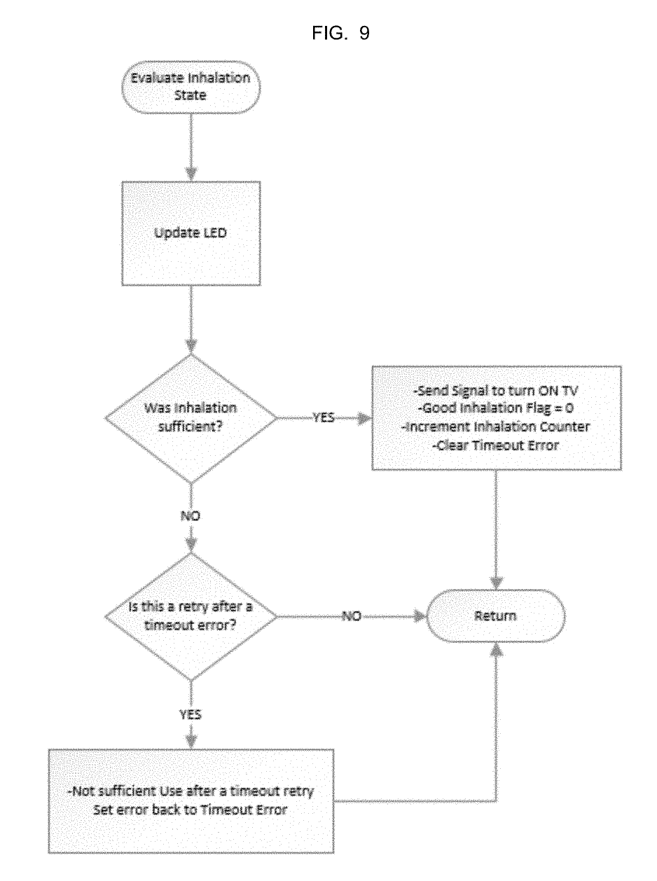

Evaluate Inhalation State. FIG. 9 shows a flowchart of the Evaluate Inhalation state. This state will inform the patient if the spirometer use has met the minimum requirement. The LED will blink green 5× if the use was sufficient and blink red 5× if the use was not sufficient. If the use was sufficient, a signal will be sent through Bluetooth to turn on power to the TELEVISION.

Send Data State. This state follows the Evaluate inhalation state and is used send inhalation data to Bluetooth module #2. This allows a device that is paired with Bluetooth module #2 (e.g., a computer, cell phone, storage device) to collect inhalation data.

Evaluate Use State

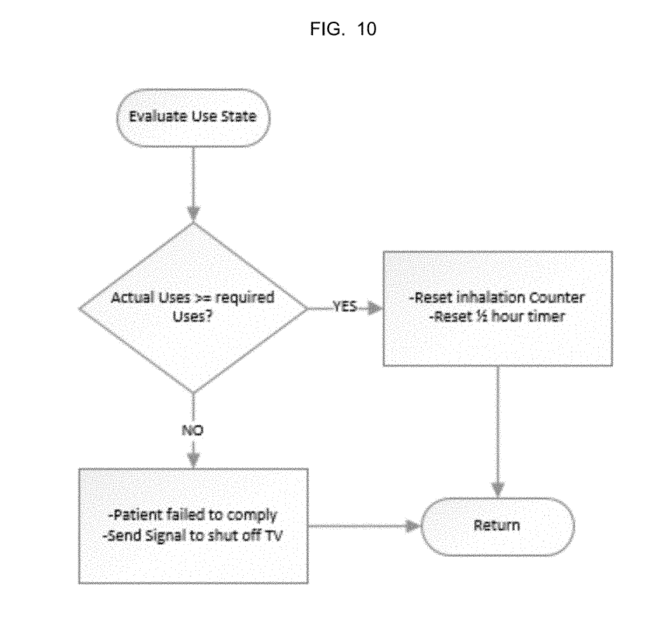

FIG. 10 shows a flowchart of the Evaluate Use state. This state will evaluate if the minimum uses per prescribed time interval have been completed. If so, the timers and counters will be reset to restart the evaluation of use period. If the minimum spirometer uses have not been completed, the signal to shut off the television will be sent through Bluetooth to the shutoff unit, and the timeout error flag will be set.

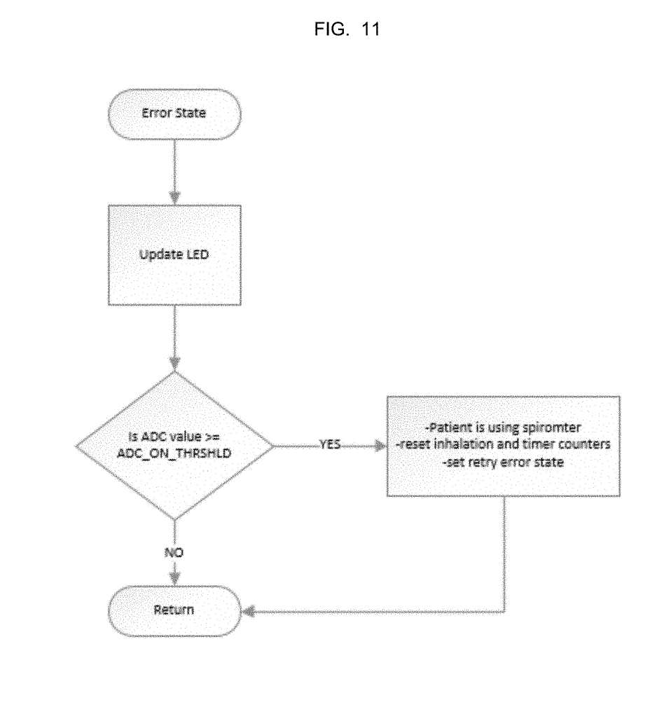

Error State. FIG. 11 shows a flowchart of the Error state. The Error state will update the LED to either solid yellow (ADC error) or solid red (timeout error). The only way to transition out of the error state is to successfully perform a spirometer inhalation. When the patient attempts to the use the spirometer, the timeout error will be set to a retry error value. This allows the software to transition through the necessary states in order to measure an inhalation, and still remain in an error state if the inhalation does not meet the minimum time requirement for a good inhalation effort (e.g., inhalation for 0.5 seconds).

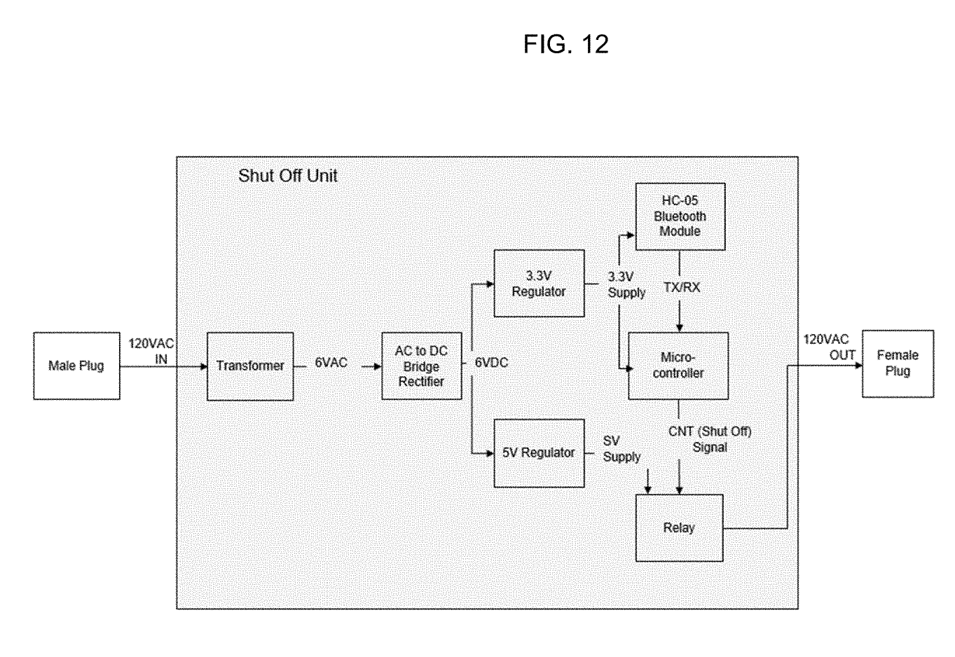

Shutoff Unit Hardware Design. A prototype Bluetooth-controlled shutoff unit was designed to work with the telecentive spirometer to turn off a patient's television when the required breaths from the spirometer are not met. FIG. 12 shows a block diagram of this prototype shutoff unit. As depicted, a three-prong male plug from a 120 VAC wall outlet is connected to the shutoff enclosure. Inside the enclosure, the 120 VAC line is split to supply power for the control circuitry and to the relay switch. The 120 VAC voltage is stepped down to 6 VAC for the control circuitry and converted to direct current (i.e., 6 VDC). This voltage is further split to supply the power requirements of the relay circuit, Bluetooth module, and microcontroller. In order to actually turn off the television, a Bluetooth signal is sent to the shutoff unit, received by the Bluetooth module in the shutoff unit, and then the information is sent through transmit and receive signals (TX/RX) to the microcontroller. The microcontroller sends the “turn on” or “shut off” signals through a control input signal to the relay. The output side of the relay has a 120 VAC line exiting the casing of the shutoff unit, and terminating in a three-prong female plug for the television.

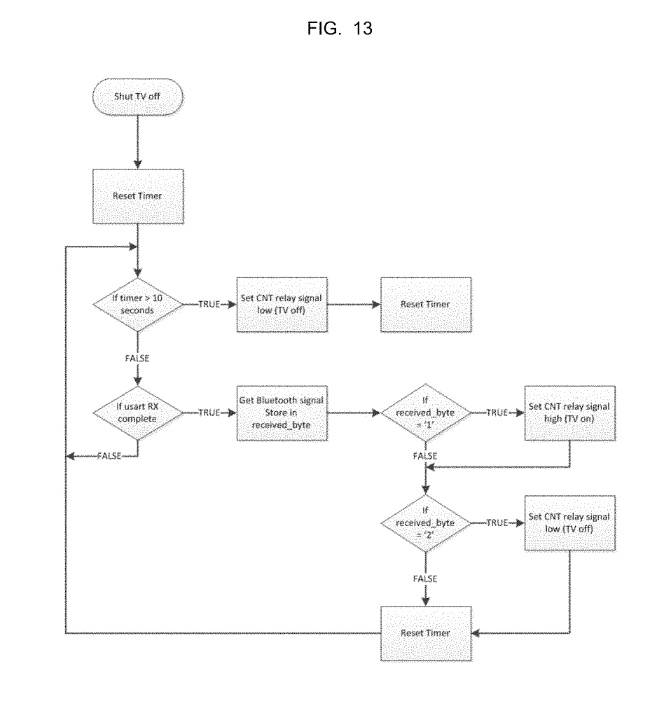

Shutoff Unit Software Design. A flowchart of the code used to program the microcontroller for the shutoff unit is shown in FIG. 13. The algorithm for the code starts by setting the control signal for the relay low (television off) to ensure the user uses the spirometer device before the television turns on, and the internal timer is initialized to 0. The code then goes into a continuous loop. In the loop, the microprocessor first checks to see if the timer is greater than 10 seconds. If this is true, the control input to the relay is set low (television off) and the timer is reset. The timer is used to ensure that a patient does not turn off the main telecentive spirometer unit to avoid the television turning off. If the user does turn off the main unit, the television shuts off after 10 seconds. If the timer is less than 10 seconds, the USART RX bit is checked for a complete received character. If this statement is false, the microprocessor starts the loop over. If the statement is true and a Bluetooth character has been sent, the character is stored in a variable called received byte. If the received byte is the character ‘1’, the control signal for the relay is set high (television on) otherwise a second check is performed. If the received byte contains the character ‘2’, the control signal for the relay is set low (television off). If a character has been sent but it is not a ‘1’ or ‘2’, this means a watchdog signal was sent to confirm the main unit and shutoff unit are connected. If any of the three characters are sent, the internal timer is reset before returning to the top of the loop.

1. A system for encouraging patient compliance with a prescribed pulmonary therapy comprising:
an incentive spirometer;
an airflow detection component operatively connected to the incentive spirometer;
a processing unit coupled to the airflow detection component, wherein the processing unit is programmed to monitor usage of the incentive spirometer and provide an indicator of patient compliance;
a communication module operatively connected to the processing unit and configured to transmit the indicator of patient compliance;
a shutoff unit configured to receive the indicator of patient compliance from the communication module and, in the case of patient non-compliance, interrupt power to an entertainment device.

2. The system of claim 1, further comprising a programming interface configured to allow a clinician to specify a set of prescribed usage requirements.

3. The system of claim 2, wherein the prescribed usage requirements comprise the number of inhalations to be performed within a time interval, and the length of said time interval.

4. The system of claim 1, wherein the communication module is further configured to transmit incentive spirometer usage data to a data storage unit.

5. The system of claim 4, wherein said incentive spirometer usage data comprises usage history data and summary statistics.

6. The system of claim 1, wherein the entertainment device is a television, radio, or gaming device.

7. The system of claim 2, further comprising a status indicator configured to provide feedback to a patient when the incentive spirometer has been used according to the prescribed usage requirements.

8. The system of claim 2, further comprising a warning indicator configured to alert a patient that shutdown of an entertainment device will occur if the prescribed usage requirements are not met.

9. A method for monitoring usage of an incentive spirometer comprising:
coupling a processing unit with a prescribed usage time interval and a prescribed number of inhalations to be exerted within said time interval to a patient's incentive spirometer;
initializing an inhalation counter in the processing unit;
starting a timer in the processing unit;
continuously monitoring incentive spirometer airflow detector for an inhalation event;
determining whether a detected inhalation event exceeds a threshold, and if so, incrementing the inhalation counter;
repetitively comparing elapsed time to the prescribed usage time interval; and
transmitting an instruction, by the processing unit, to a shutoff unit when the elapsed time exceeds the prescribed usage time interval, provided that the value of a usage counter is not equal to or greater than the prescribed number of inhalations, wherein reception of the instruction by the shutoff unit causes interruption of power to an entertainment device.

10. The method of claim 9, wherein the threshold is defined as an inhalation signal of a specified duration.

11. The method of claim 10, wherein the specified duration is 0.5 seconds.

12. The method of claim 9, wherein the entertainment unit is a television, radio, or gaming device.
